(19) United States
(12) Patent Application Publication
(10) Pub. No.: US 2014/0301952 A1
(43) Pub. Date: Oct. 9, 2014

(54) MEASUREMENT OF BODY FLUID VOLUMES

(71) Applicant: PHARMACOPHOTONICS, INC. D/B/A FAST DIAGNOSTICS, Indianapolis, IN (US)

(72) Inventors: Bruce A. Molitoris, Indianapolis, IN (US); Exing Wang, Carmel, IN (US); Ruben M. Sandoval, JR., Indianapolis, IN (US)

(21) Appl. No.: 14/309,969

(22) Filed: Jun. 20, 2014

Related U.S. Application Data
(63) Continuation-in-part of application No. 13/318,097, filed on Apr. 18, 2012.
(60) Provisional application No. 61/174,100, filed on Apr. 30, 2009.

(51) Int. Cl. A61K 49/00; G01N 33/487

(52) U.S. Cl. CPC A61K 49/0054; G01N 33/487; G06F 19/10
USPC 424/9.6; 73/61.59; 702/19

(57) ABSTRACT

The present invention relates generally to measurement of body fluid volumes in an animal subject. The body fluid volumes of interest include extracellular fluid volume (ECFV), total vascular plasma volume (TVPV) and interstitial fluid volume (IFV). The methods are especially beneficial for subjects suffering from renal failure and particularly those undergoing renal dialysis. ECFV can be measured by administering a first molecule which is non-metabolized and permeable to vessel walls of the vascular system, wherein the first molecule is distributed within the total vascular space as well as the interstitial space. TVPV can be measured by administering a second molecule which is non-metabolized and impermeable to vessel walls of the vascular system, wherein the second molecule is distributed within only the vascular space. IFV can then be calculated using the equation IFV=ECFV−TVPV.

### CROSS REFERENCE TO RELATED APPLICATIONS

The present application is a continuation-in-part of U.S. patent application Ser. No. 13/318,097, filed Apr. 18, 2012, which is a national stage application of PCT/US2010/032997, filed Apr. 29, 2010, which claims priority to U.S. provisional application No. 61/174,100, filed Apr. 30, 2009. The contents of the above-identified applications are incorporated herein by reference.

### BACKGROUND OF THE INVENTION

The present invention is related generally to measurement of body fluid volumes in an animal subject. The body fluid volumes of interest include extracellular fluid volume (ECFV), total vascular plasma volume (TVPV) and interstitial fluid volume (WV). The methods are especially beneficial for subjects suffering from renal failure, and particularly those undergoing renal dialysis.

The present invention discloses methods and apparatus for measuring the various body fluid volumes in an animal, particularly in animals with renal failure, and more particularly in a renal dialysis patients. The body fluid volumes of interest in the present application are extracellular fluid volume (ECFV), total vascular plasma volume (TVPV) and interstitial fluid volume (WV).

Body fluid volume status is a critical metric in the management of many chronic and acute medical conditions. Volume status is a key determinant in drug dosing, pharmacokinetics, blood pressure and organ perfusion. Volume status and volume management are most critical in indications or conditions such as, but are not limited to, end stage renal disease (ESRD), hypertension, congestive heart failure, septic shock and hypovolemia, acute kidney injury and chronic kidney disease (CKD), hypertension, syncope, acute blood loss, pre-surgical screening, orthostatic hypotension and anemia in cancer or HIV. In addition, evaluating total vascular plasma volume and interstitial fluid volume in dialysis patients has very important implications especially with regard to removal of volume while on dialysis. This is clinically very important for control of blood pressure and clinical outcomes in patients with end stage renal disease (ESRD) who all require chronic forms of dialysis or renal replacement therapy (RRT) for volume removal.

The importance of volume status and volume management in dialysis patients has been discussed by Agarwal R. et al. (“Diagnostic Utility of Blood Volume Monitoring in Hemodialysis Patients”, Am J of Kidney Diseases (2008) 51: 242-254), Rodriguez H. J. et al. (“Assessment of Dry Weight by Monitoring Changes in Blood Volume During Hemodialysis using Crit-Line”, Kidney International (2005) 68, 854-861), Kraemer M. et al. (“Detection Limit of Methods to Assess Fluid Status Changes in Dialysis Patients”, Kidney International (2006) 69: 1609-1620), and Dasselaar J. J. et al. (“Measurment of Relative Blood Volume Changes During Haemodialysis: Merits and Limitations”, Nephrol Dial Transplant (2005) 20: 2043-2049).

A commonly used technique for estimating the TVPV is based on the concept of the indicator dilution technique in which an indicator molecule is mixed and distributed into an unknown volume. An identical amount of the indicator molecule is placed into a known volume. The unknown volume can be measured by comparing the concentration of the indicator between the known and unknown volume. A common indicator molecule that is being used is albumin labeled with various dyes, such as radioactive iodine (I¹²⁵ or I¹³¹), or the fluorescent dye indocyanine green (ICG). For example, Daxor Corporation (New York, N.Y.) has developed a device for measuring blood volume using albumin labeled with I¹³¹ as the tracer indicator.

The use of ICG-labeled albumin as the tracer indicator has been disclosed by Mitra, S. et al. (“Serial Determinations of Absolute Plasma Volume with Indocyanine Green During Hemodialyais,”, J Am Soc of Nephrology (2003) 14(9): 2345-51). In this method, ICG-labeled albumin was measured by near infra-red absorption of the molecule. Functionally, there is little difference between the use of ICG when compared to I¹³¹, as both quickly bind to albumin in the bloodstream. The main distinguishing characteristics are the relatively short half life of ICG as compared to I¹³¹ and the beneficial safety profile of ICG. ICG is already approved for human use by the United States Food And Drug Administration (FDA). The short half life of ICG allows for multiple tests to be conducted with rapid succession. However, utility of the ICG method has been limited by many of the same factors as the iodine-based testing. Though the time period for collecting samples of ICG is much shorter than the radioactive test, it becomes all the more important to make certain that sampling is conducted at precise time intervals. Therefore, it is a very labor intensive method. Another drawback in the use of labeled albumin, in the dilution technique to measure plasma volume, is that albumin also “leaks” and distributes to the interstitial fluid. Under physiologic conditions, albumin “leaks” into the interstitial space at a rate of about 5% per hour. This rate increases to 15% per hour in patients with septic shock (see U.S. Pat. No. 6,355,624). Thus, albumin does not measure the true TVPV or plasma volume, but rather it measures the combination of the TVPV and the IFV.

Another method that is used to measure body fluid volumes is the use of bioimpedence spectroscopy. This approach has been discussed by Zhu et al. (“Segment-Specific Resistivity Improves Body Fluid Volume Estimates from Bioimpedence Spectroscopy in Hemodialysis Patients”, J Appl Physio (2006) 100: 717-724), De Lorenzo A. et al. (“Predicting Body Cell Mass With Bioimpedance by Using Theoretical Methods: a Technological Review”, J Appl Physiology (1997) 82: 1542-1558), and Kuhlmann, M. K. et al. (“Bioimpedence, Dry Weight and Blood Pressure Control: New Methods and Consequences”, Current Opinion in Nephrology and Hypertension (2005) 14: 543-549). However, this technique is too difficult and impractical to perform.

Therefore, there is a clinical need to develop a minimally invasive method to accurately and inexpensive quantify these body fluid volumes. The present invention is provided to solve the problems discussed above and other problems, and to provide advantages and aspects not provided by prior techniques. A full discussion of the features and advantages of the present invention is deferred to the following detailed description.

### SUMMARY OF THE INVENTION

One aspect of the present invention is directed to methods for measuring extraceullar fluid volume (ECFV) in an animal with renal failure. The method comprises: (a) administering a sufficient amount (A₁) of a first molecule to the vascular system of the animal wherein the first molecule is non-metabolized and permeable to vessel walls of the vascular system; (b) allowing the first molecule to reach a first equilibrium steady state concentration (C₁) in the vascular system of the animal; (c) measuring the C₁ in the vascular system of the animal; and (d) calculating the ECFV using the equation: ECFV=A₁/C₁. The first molecule may be administered by intravenous injection of an injectate containing the first molecule. The intravenous injection can be bolus or continuous infusion. Alternatively, the first molecule may be administered by inhalation.

In an embodiment, the first molecule has a molecular size of from about 1 kDa to about 20 kDa. In another embodiment, the first molecule is dextran. In yet another embodiment, the first molecule is labeled with a first fluorescent dye and the first molecule is detected and quantified by the fluorescence intensity of the molecule. The first fluorescent dye can be selected from, but not limited to, xanthene dye, CAL FLOUR®, ALEXA FLOUR®, carbocyanine, fluorescein, fluorescein isothiocyanate (FITC), carboxy fluoresecein, cyanine, rhodamine, tetramethylrhodamine (Tamra), tetramethyl rhodamine isothiocyanate (TRITC), X rhodamine isothiocyanate (XRITC), TEXAS RED®, and indocyanine green (ICG).

Preferably, the first molecule is a rhodamine dye which can be conjugated to a dextran molecule to form a single isomeric conjugation product. In this embodiment, the rhodamine dye contains only a single functional group on the rhodamine molecule for conjugation so that the conjugation product is a single isomeric conjugation product. Suitable rhodamine dyes are disclosed in U.S. published patent application no. 2013/0096309, published Apr. 18, 2013, the disclosure of which is incorporated by reference herein in its entirety.

Measurement of the concentration of the first molecular dye in the vascular system can be performed in vitro or in vivo. In the in vitro method, a sample of blood is drawn from the animal after the first molecule has reached a steady state equilibrium concentration in the vascular system of the animal. A plasma or serum supernatant is prepared from the blood sample by a method such as, but not limited to, centrifugation or filtration. The fluorescence intensity of the first molecule is measured in the supernatant. In the in vivo method, the fluorescence intensity of the first molecule is measured directly in vivo within the vascular system of the animal without having to remove a blood sample from the animal. A preferred method for in vivo measurement of the first molecule is to use a first molecule labeled with a first fluorescent dye.

A second aspect of the invention is directed to methods for determining extraceullar fluid volume (ECFV) in an animal with renal failure comprising: (a) providing a first injectate having a volume V₁ containing a first molecule labeled with a first fluorescent dye having a first excitation wavelength and a first emission wavelength, wherein the first molecule is non-metabolized and permeable to vessel walls of the vascular system and the first injectate has a first emission fluorescence intensity of F₁; (b) administering the first injectate to the vascular system of the animal; (c) allowing the first molecule to reach a first equilibrium steady state concentration in the vascular system of the animal; (d) exciting the first molecule with the first excitation wavelength in vivo in the vascular system of the animal; (e) measuring the second emission fluorescence intensity F₂ of the first molecule in vivo in the vascular system of the animal; and (f) calculating the ECFV using the equation: ECFV=(F₁*V₁)/F₂.

A third aspect of the invention is directed to methods for determining total vascular plasma volume (TVPV) in an animal comprising: (a) administering a sufficient amount (A₂) of a second molecule to the vascular system of the animal, wherein the second molecule is non-metabolized and impermeable to vessel walls of the vascular system; (b) allowing the second molecule to reach a second equilibrium steady state concentration in the plasma within the vascular system of the animal; (c) measuring the second equilibrium steady state concentration (C₂) of the second molecule; and calculating the TVPV using the equation: TVPV=A₂/C₂.

In an embodiment, the second molecule has a molecular size of from about 70 kDa to about 500 kDa, preferably from about 70 kDa to about 250 kDa, and most preferably about 150 kDa. In another embodiment, the second molecule is a dextran. In yet another embodiment, the second molecule is labeled with a second fluorescent dye and the second molecule is detected by the emission fluorescence intensity of the molecule. The second fluorescent dye can be selected from, but not limited to, xanthene dye, CAL FLOUR®, ALEXA FLOUR®, carbocyanine, fluorescein, fluorescein isothiocyanate (FITC), carboxy fluoresecein, cyanine, rhodamine, tetramethylrhodamine (Tamra), tetramethyl rhodamine isothiocyanate (TRITC), X rhodamine isothiocyanate (XRITC), TEXAS RED®, and indocyanine green (ICG).

Preferably, the second molecule is a rhodamine dye which can be conjugated to a dextran molecule to form a single isomeric conjugation product. In this embodiment, the rhodamine dye contains only a single functional group on the rhodamine molecule for conjugation so that the conjugation product is a single isomeric conjugation product. Suitable rhodamine dyes are disclosed in U.S. published patent application no. 2013/0096309, published Apr. 18, 2013, the disclosure of which is incorporated by reference herein in its entirety.

Measurement of the concentration of the second molecule in the vascular system can be performed in vitro or in vivo. In the in vitro method, a sample of blood is drawn from the animal after the second molecule has reached a steady state equilibrium concentration in the vascular system of the animal. A plasma or serum supernatant is prepared from the blood sample by a method such as, but not limited to, centrifugation or filtration. The concentration of the second molecule is measured in the plasma or serum supernatant. In the in vivo method, the second molecule is measured directly in vivo within the vascular system of the animal without having to remove a blood sample from the animal. A preferred method for in vivo measurement of the second molecule is to use a second molecule labeled with a second fluorescent dye.

A forth aspect of the invention is directed to methods for determining total vascular plasma volume (TVPV) in an animal comprising: (a) providing a second injectate having a volume V₂ containing a second molecule labeled with a second fluorescent dye having a second excitation wavelength and a second emission wavelength, wherein the second molecule is non-metabolized and impermeable to vessel walls of the vascular system and the second injectate has a third emission fluorescence intensity of F₃; (b) administering the second injectate to the vascular system of the animal; (c) allowing the second molecule to reach a second equilibrium steady state concentration in the vascular system of the animal; (d) exciting the second molecule with the second excitation wavelength in vivo in the vascular system of the animal; (e) measuring the forth emission fluorescence intensity F₄ of the second molecule in vivo in the vascular system of the animal; and (f) calculating the TVPV using the equation: TVPV=(F₃*V₂)/F₄.

A fifth aspect of the invention is directed to a method for determining the interstitial fluid volume (IFV) in an animal comprising: (a) determining the extracellular fluid volume (ECFV) of the animal; (b) determining the total vascular plasma volume (TVPV) of the animal; and (c) calculating the IFV of the animal using the equation: IFV=ECFV−TVPV.

A sixth aspect of the invention is directed to methods for simultaneously measuring extracellular fluid volume (ECFV) and total vascular plasma volume (TVPV) in an animal with renal failure comprising: (a) providing an injectate containing a known amount A₁ of a first molecule and a known amount A₂ of a second molecule, wherein the first molecule is non-metabolized and permeable to vessel walls of the vascular system of the animal and the second molecule is non-metabolized and impermeable to vessel walls of the vascular system of the animal; (b) administering the injectate into the vascular system of the animal; (c) allowing the first molecule to reach a first equilibrium steady state concentration C₁ and the second molecule to reach a second equilibrium steady state concentration C₂; (d) measuring C₁ and C₂ in the vascular system of the animal; and (e) calculating ECFV using the equation ECFV=A₁/C₁ and TVPV using the equation TVPV=A₂/C₂.

Measurement of the concentration of the first molecule and the second molecule in the vascular system can be performed in vitro or in vivo. In the in vitro method, a sample of blood is drawn from the animal after the first molecule and the second molecule have each reached a steady state equilibrium concentration in the vascular system of the animal. A plasma or serum supernatant is prepared from the blood sample by a method such as, but not limited to, centrifugation or filtration. The concentration of the first molecule and the second molecule is measured in the supernatant. In the in vivo method, the first molecule and the second molecule are measured directly in vivo within the vascular system of the animal without having to remove a blood sample from the animal. A preferred method for in vivo measurement of the first molecule and the second molecule is to use a first molecule labeled with a first fluorescent dye and a second molecule labeled with a second fluorescent dye that is different from the first dye.

The method may further comprise an additional step of calculating the interstitial fluid volume (IFV) using the equation: IFV=ECFV−TVPV.

An apparatus for determining the ECVF and TVPV using these methods may comprise: (a) means for providing the injectate to the vascular system of the animal; (b) means for measuring C₁ and C₂ in vivo in the vascular system of the animal; (c) means for calculating ECFV and TVPV; and (d) means for displaying the calculated values of ECFV and TVPV. Optionally, the apparatus may further comprise means for calculating IFV and displaying the calculated value of IFV. The apparatus may be a stand alone unit or incorporated into a hemodialysis device.

A seventh aspect of the invention is directed to methods for simultaneously measuring extracellular fluid volume (ECFV) and total vascular plasma volume (TVPV) in an animal with renal failure comprising: (a) providing an injectate having a volume V containing a first molecule and a second molecule, wherein the first molecule (i) is labeled with a first fluorescent dye having a first excitation wavelength and a first emission wavelength, (ii) is non-metabolized and permeable to vessel walls of the vascular system of the animal, and (iii) has a first emission fluorescence intensity of F₁, and wherein the second molecule (i) is labeled with a second fluorescent dye having a second excitation wavelength and a second emission wavelength, (ii) is non-metabolized and impermeable to vessel walls of the vascular system of the animal and (iii) has a second emission fluorescence intensity of F₂; (b) administering the injectate into the vascular system of the animal; (c) allowing the first molecule and the second molecule to each reach equilibrium steady state concentrations within the vascular system of the animal; (d) exciting the first molecule in vivo in the vascular system of the animal with a first excitation light source having a first excitation wavelength and exciting the second molecule in vivo in the vascular system of the animal with a second excitation light source having a second excitation wavelength; (e) measuring the third emission fluorescence intensity F₃ from the first molecule in vivo in the vascular system of the animal and measuring the forth emission fluorescence intensity F₄ from the second molecule in vivo in the vascular system of the animal; and (f) calculating the ECFV using the equation ECFV=(F₁*V)/F₃ and the TVPV using the equation TVPV=(F₂*V)/F₄.

The method may further comprise an additional step of calculating the interstitial fluid volume (WV) using the equation: IFV=ECFV−TVPV.

Other features and advantages of the invention will be apparent from the following specification taken in conjunction with the following drawings.

### BRIEF DESCRIPTION OF THE DRAWINGS

FIG. 1 shows a decay curve (∘) of the fluorescence from the larger fluorescent marker 150-kDa FITC-dextran which was administered to a bilaterally anephric rat as described in Example 1. Also shown is the smoothed fluorescence curve ( - - - ) of the fluorescence from the 150-kDa FITC-dextran as well as a decay curve of the ratio of the fluorescence from the 3-kDa Texas Red-dextran to that of the 150-kDa FITC dextran ( - - - - - - ).

### DETAILED DESCRIPTION OF THE INVENTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The present invention is related generally to the measurement of body fluid volumes in an animal subject. The body fluid volumes of interest include extracellular fluid volume (ECFV), total vascular plasma volume (TVPV) and interstitial fluid volume (IFV). The methods are especially beneficial for subjects suffering from renal failure and particularly those undergoing renal dialysis. The animal subject may be a mammalian subject, and the mammalian subject may be a human. The renal failure may be acute or chronic. Acute renal failure may be due to acute renal injury, and chronic renal failure may be due to late stage renal disease. Renal dialysis can be hemodialysis or peritoneal dialysis if the abdominal cavity is dry. The renal failure may also be temporary or permanent.

In brief, ECFV can be measured by administering a first molecule which is non-metabolized and permeable to vessel walls of the vascular system wherein the first molecule is distributed within the total vascular space as well as the interstitial space. TVPV can be measured by administering a second molecule which is non-metabolized and impermeable to vessel walls of the vascular system wherein the second molecule is distributed within only the vascular space. IFV can then be calculated using the equation IFV=ECFV−TVPV.

What is meant by total vascular plasma volume (TVPV) as used in the present application is the amount of plasma volume contained within the entire vascular space including arterial, venous and capillary spaces. The TVPV does not include the volume contributed by the blood cells, such as the red blood cells. TVPV may also be referred to as the Plasma Volume (PV).

What is meant by interstitial fluid volume (IFV) as used in the present application is the amount of volume extra vascular and surrounding cells as well as collections of fluid such as ascites or pleural fluid. IFV is a good indicator for capillary leakage. Expanded IFV is indicative that fluid is leaking from the vascular system and accumulating into the interstitial space which results in edema.

The extracellular fluid volume (ECFV) as used in the present application is the sum of the TVPV and IFV. The relationship between these volumes can, therefore, be represented by the following equation:

ECFV=TVPV+IFV (1)

Total blood volume (TBV) can be estimated from the TVPV by adding TVPV and the volume contributed by the blood cells, which can be determined from the Hematocrit (Hct) or from the Packed Cell Volume (PCV).

One aspect of the present invention is directed to methods for measuring extraceullar fluid volume (ECFV) in an animal with renal failure. The method comprises: (a) administering a sufficient amount (A₁) of a first molecule to the vascular system of the animal wherein the first molecule is non-metabolized and permeable to vessel walls of the vascular system; (b) allowing the first molecule to reach a first equilibrium steady state concentration (C₁) in the vascular system of the animal; (c) measuring the C₁ in the vascular system of the animal; and (d) calculating the ECFV using the equation: ECFV=A₁/C₁.

Another aspect of the present invention is directed to methods for determining total vascular plasma volume (TVPV) in an animal comprising: (a) administering a sufficient amount (A₂) of a second molecule to the vascular system of the animal, wherein the second molecule is non-metabolized and impermeable to vessel walls of the vascular system; (b) allowing the second molecule to reach a second equilibrium steady state concentration in the plasma within the vascular system of the animal; (c) measuring the second equilibrium steady state concentration (C₂) of the second molecule; and (d) calculating the TVPV using the equation: TVPV=A₂/C₂.

Once the ECFV and the TVPV are determined, interstitial fluid volume (IFV) can be calculated using the equation:

IFV=ECFV−TVPV (2)

What is meant by a sufficient amount of the first molecule or the second molecule is that the molecule is above the detection limit using an appropriate analytical technique after the molecule has reached equilibrium following distribution. The appropriate analytical method depends on the properties and characteristics of the molecule. Examples of commonly used analytical methods include, but are not limited to, absorption spectroscopy, fluorescence, adsorption, and radioactive activity of the molecule.

The time required for the first molecule or the second molecule to reach its respective steady state equilibrium concentration depends on the particular molecule and the animal species. Such time for reaching equilibrium can easily be determined by dosing the animal with the molecule, and monitoring the molecule in the vascular system of the animal over time. Initially, the concentration of the molecule rises in the vascular system, which represents a mixing phase of the molecule in the vascular system. Eventually, the concentration of the molecule reaches an equilibrium steady state in the vascular system when the concentration plateaus. The beginning of the plateau of the concentration of the molecule marks the end of the mixing phase. An example of such a method is described in Example 1 below. This equilibrium time is relatively constant for a specific molecule and a specific animal species so that once such time is determined for the molecule and the animal species, the value can be used for the same molecule and the same animal species without having to determine the value again. In human beings, the equilibrium time is about 10 to 15 minutes for most molecules. However, in certain disease states, such as congestive heart failure, it may take a longer time to reach equilibrium.

The first molecule or the second molecule may be administered by a suitable method such as intravenous injection of an injectate containing the first molecule and/or the second molecule. The intravenous injection can be bolus or continuous infusion over a period of time.

What is meant by “non-metabolized” is that the molecule is not significantly metabolized by the animal during the time in which the measurements are performed.

What is meant by “permeable to vessel walls” refers to that the molecule can cross the vessel walls. This movement of the molecule can be a passive method without requiring energy, e.g. diffusion, or an active method requiring energy, e.g. active transport.

Similarly, “impermeable to vessel walls” refers to the concept that the molecule cannot cross the vessel walls either through a passive process or an active process.

In an embodiment, the first molecule has a molecular size of from about 1 kDa to about 20 kDa. In another embodiment, the second molecule has a molecule size of from about 70 kDa to about 500 kDa, preferably from about 100 kDa to about 200kDa, and most preferably about 150 kDa. In yet another embodiment, the first or the second molecule are dextrans. In a further embodiment, the first molecule or the second molecule is a fluorescent molecule. In yet a further embodiment, the first molecule is a dextran labeled with a first fluorescent dye having a first excitation wavelength and a first emission wavelength. In still a further embodiment, the second molecule is a dextran labeled with a second fluorescent dye having a second excitation wavelength and a second emission wavelength. The first or second fluorescent dye can be selected from, but not limited to, xanthene dye, CAL FLOUR®, ALEXA FLOUR®, carbocyanine, fluorescein, fluorescein isothiocyanate (FITC), carboxy fluoresecein, cyanine, rhodamine, tetramethylrhodamine (Tamra), tetramethyl rhodamine isothiocyanate (TRITC), X rhodamine isothiocyanate (XRITC), TEXAS RED®, and indocyanine green (ICG). The first or second fluorescent dye can be the same or different.

Preferably, the first and second molecules are rhodamine dyes which can be conjugated to dextran molecules to form single isomeric conjugation products. In this embodiment, the rhodamine dyes contain only a single functional group on the rhodamine molecule for conjugation, so that the conjugation product is a single isomeric conjugation product. Suitable rhodamine dyes are disclosed in U.S. published patent application no. 2013/0096309, published Apr. 18, 2013, the disclosure of which is incorporated by reference herein in its entirety.

The first molecule may be administered separately from the second molecule, or both molecules can be administered simultaneously. Administration is advantageously accomplished using a catheter for injection into the vascular system of a subject.

Measurement of the concentration of the first molecule or the second molecule in the vascular system can be performed in vitro or in vivo. In the in vitro method, a sample of blood is drawn from the animal after the first molecule or the second molecule has reached a steady state equilibrium concentration in the vascular system of the animal. A plasma or serum supernatant of the blood is prepared from the blood sample by a method which removes the blood cells from the blood, such as, but not limited to, centrifugation or filtration. These separation methods are well known to those skilled in the art and are routinely practiced in the laboratory. The supernatant represents the plasma of the blood. The concentration of the first molecule or the second molecule is measured in the supernatant by an appropriate detection method such as absorption spectroscopy or fluorescence. In the in vivo method, the first or the second molecule is measured directly in vivo within the vascular system of the animal without having to remove a blood sample from the animal. A preferred method for in vivo measurement of a molecule is to use a molecule labeled with a fluorescent dye. An example of an in vivo measurement of a fluorescent molecule in the vascular system of the animal has been disclosed in a pending U.S. Pat. No. 12/425,827 which is incorporated herein by reference and made a part of the present application. The method is applicable to measuring one or more fluorescent molecules simultaneously in vivo.

In the embodiment directed to methods for measuring ECVF in an animal with renal failure in which the first molecule is a fluorescent molecule, the method may comprise: (a) providing a first injectate containing the first molecule having a first excitation wavelength and a first emission wavelength, the first injectate having a volume of V₁ wherein the first molecule is non-metabolized and permeable to vessel walls of the vascular system of the animal; (b) measuring a first emission fluorescence intensity F₁ of the first molecule in the first injectate; (c) administering the first injectate into the vascular system of the animal; (d) allowing the first molecule to reach a steady state equilibrium concentration; (e) measuring a second emission fluorescence intensity F₂ of the first molecule in the vascular system; and (f) calculating the ECVF using the equation: ECVF=(F₁*V₁)/F₂. F₂ may be measured in vitro or in vivo.

In the embodiment in which F₂ is measured in vivo, the method comprises: (a) providing a first injectate having a volume V₁ containing a first molecule labeled with a first fluorescent dye having a first excitation wavelength and a first emission wavelength, wherein the first molecule is non-metabolized and permeable to vessel walls of the vascular system, and the first injectate has a first emission fluorescence intensity of F₁; (b) administering the first injectate to the vascular system of the animal; (c) allowing the first molecule to reach a first equilibrium steady state concentration in the vascular system of the animal; (d) exciting the first molecule with the first excitation wavelength in vivo in the vascular system of the animal; (e) measuring the second emission fluorescence intensity F₂ of the first molecule in vivo in the vascular system of the animal; and (f) calculating the ECFV using the equation: ECFV=(F₁*V₁)/F₂.

Similarly, in the embodiment directed to methods for measuring TVPV in an animal in which the second molecule is a fluorescent molecule, the method may comprise: (a) providing a second injectate containing the second molecule having a second excitation wavelength and a second emission wavelength, the second injectate having a volume of V₂ wherein the second molecule is non-metabolized and impermeable to vessel walls of the vascular system of the animal; (b) measuring a third emission fluorescence intensity F₃ of the second molecule in the second injectate; (c) administering the second injectate into the vascular system of the animal; (d) allowing the second molecule to reach a steady state equilibrium concentration; (e) measuring a forth emission fluorescence intensity F₄ of the second molecule in the vascular system; and (f) calculating the ECVF using the equation: TVPV=(F₃*V₂)/F₄. F₄ may be measured in vitro or in vivo.

In the embodiment in which F₄ is measured in vivo, the method comprises: (a) providing a second injectate having a volume V₂ containing a second molecule labeled with a second fluorescent dye having a second excitation wavelength and a second emission wavelength, wherein the second molecule is non-metabolized and impermeable to vessel walls of the vascular system and the second injectate has a third emission fluorescence intensity of F₃; (b) administering the second injectate to the vascular system of the animal; (c) allowing the second molecule to reach a second equilibrium steady state concentration in the vascular system of the animal; (d) exciting the second molecule with the second excitation wavelength in vivo in the vascular system of the animal; (e) measuring the forth emission fluorescence intensity F₄ of the second molecule in vivo in the vascular system of the animal; and (f) calculating the TVPV using the equation: TVPV=(F₃*V₂)/F₄.

A further aspect of the invention is directed to methods for simultaneously measuring extracellular fluid volume (ECFV) and total vascular volume (TVPV) in an animal with renal failure comprising: (a) providing an injectate containing a known amount Aₗ of a first molecule and a known amount A₂ of a second molecule, wherein the first molecule is non-metabolized and permeable to vessel walls of the vascular system of the animal and the second molecule is non-metabolized and impermeable to vessel walls of the vascular system of the animal; (b) administering the injectate into the vascular system of the animal; (c) allowing the first molecule to reach a first equilibrium steady state concentration C₁ and the second molecule to reach a second equilibrium steady state concentration C₂; (d) measuring C₁ and C₂ in the vascular system of the animal; and (e) calculating ECFV using the equation ECFV=A₁/C₁, and TVPV using the equation TVPV=A₂/C₂.

Measurement of the concentration of the first molecular and the second in the vascular system can be performed in vitro or in vivo. In the in vitro method, a sample of blood is drawn from the animal using, for example, a catheter after the first molecule and the second molecule have each reached a steady state equilibrium concentration in the vascular system of the animal. A plasma or serum supernatant is prepared from the blood sample by a method such as, but not limited to, centrifugation or filtration. The concentration of the first molecule and the second molecule is measured in the supernatant. In the in vivo method, the first molecule and the second molecule are measured directly in vivo within the vascular system of the animal without having to remove a blood sample from the animal. A preferred method for in vivo measurement of the first molecule and the second molecule is to use a first molecule labeled with a first fluorescent dye and a second molecule labeled with second fluorescent dye.

The method may further comprise an additional step of calculating the interstitial fluid volume (IFV) using the equation: IFV=ECFV−TVPV.

An apparatus for determining the ECFV and TVPV using these methods may comprise: (a) means for providing the injectate to the vascular system of the animal; (b) means for measuring C₁ and C₂ in vivo in the vascular system of the animal; (c) means for calculating ECFV and TVPV; and (d) means for displaying the calculated values of ECFV and TVPV. Optionally, the apparatus may further comprise means for calculating IFV and displaying the calculated value of IFV. The apparatus may be a stand alone unit or incorporated into a hemodialysis device.

Yet another aspect of the invention is directed to methods for simultaneously measuring extracellular fluid volume (ECFV) and total vascular plasma volume (TVPV) in an animal with renal failure comprising: (a) providing an injectate having a volume V containing a first molecule and a second molecule, wherein the first molecule (i) is labeled with a first fluorescent dye having a first excitation wavelength and a first emission wavelength, (ii) is non-metabolized and permeable to vessel walls of the vascular system of the animal, and (iii) has a first emission fluorescence intensity of F₁, and wherein the second molecule (i) is labeled with a second fluorescent dye having a second excitation wavelength and a second emission wavelength, (ii) is non-metabolized and impermeable to the vessel walls of the vascular system of the animal, and (iii) has a second emission fluorescence intensity of F₂; (b) administering the injectate into the vascular system of the animal; (c) allowing the first molecule and the second molecule to each reach an equilibrium steady state concentration within the vascular system of the animal; (d) exciting the first molecule in vivo in the vascular system of the animal with a first excitation light source having a first excitation wavelength, and exciting the second molecule in vivo in the vascular system of the animal with a second excitation light source having a second excitation wavelength; (e) measuring the third emission fluorescence intensity F₃ from the first molecule in vivo in the vascular system of the animal, and measuring the forth emission fluorescence intensity F₄ from the second molecule in vivo in the vascular system of the animal; and (f) calculating the ECFV using the equation ECFV=(F₁*V)/F₃ and the TVPV using the equation TVPV=(F₂*V)/F₄.

The method may further comprise an additional step of calculating the interstitial fluid volume (IFV) using the equation: IFV=ECFV−TVPV.

### EXAMPLES

### Example 1

### Measurement of TVPV and ECFV in Bilaterally Anephric Rats

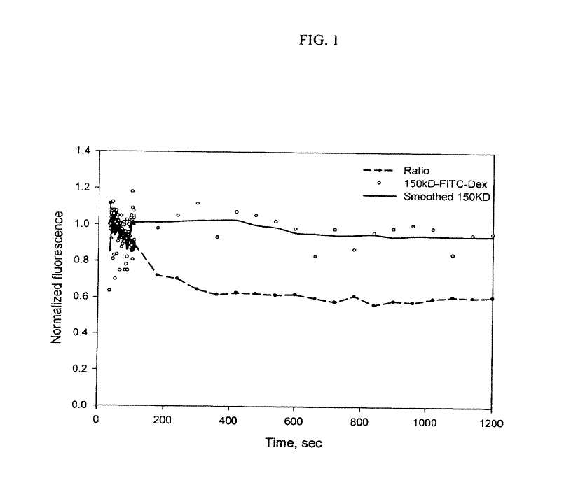

The example shown here was a test conducted on a bilaterally anephric rat, which was infused with a mixture of 3 kDa Texas Red-dextran and 150 kDa FITC-dextran. The dynamic plasma fluorescence intensity was obtained by in vivo two-photon liver imaging of vascular plasma. Only the vascular plasma-containing regions in each image were included for calculation. The decay curve of the fluorescence intensity of the 150-kDa FITC-dextran, as well as the decay curve of the ratio of the fluorescence intensity of the Texas Red-dextran to that of the FITC-dextran after the infusion is shown in FIG. 1. Using the ratio rather than the 3 kDa Texas Red dextran or the 150 kDa FITC-dextran signal directly helped reduce the signal fluctuation caused by focus movement during imaging since the same fluctuation showed up in both channels.

To test if the volumes determined by this method agree with expected values, a mixture of 3 KDa TexasRed-dextran and 150 kDa FITC-dextran was injected into two bilaterally anephric rats. Blood was drawn from the animals 15 minutes after the infusion. According to the FIG. 1, this should be more than enough time for the dextrans to become equilibrated between the vascular and the interstitial spaces. The blood plasma was then separated by centrifuge. Fluorescence was measured using a spectrophotometer. TVPV and ECFV from each rat were determined as described above. The measured volumes along with estimated plasma volumes by body weight are shown in the following table.

TABLE 1
Measured and Estimated Plasma Volumes in Anephric Rats

| | Measured | Estimated | Measured | Calculated |
|---|---|---|---|---|
| | TVPV (ml) | TVPV (ml) | ECFV (ml) | IFV |
| Rat 1 | 8.30 | 7.95 | 22.94 | 14.64 |
| Rat 2 | 6.32 | 6.77 | 18.12 | 11.80 |

Estimated TVPV values were obtained from a method described by Altman P. L. (“Blood and Other Body Fluids”, Fed of Am Societies for Experimental Biology (1961) Washington, D.C.), and Yu W. et al. (“Rapid Determinations of Renal Filtration Function using an Optical Ratiometric Image Approach”, Am J Physiology-Renal Physiology (2007) 292(6): F1873-80).

IFV can be calculated from the measured TVPV and ECFV using the equation IFV=ECFV−TVPV.

### Example 2

### Anticipated Minimally Invasive Method for Measuring Fluid Volumes in a Patient with Renal Failure

A minimally invasive method for measuring TVPV, ECFV and TV in a patient with renal failure comprises a small dextran (molecule size of about 1 kDa to about 20 kDa) labeled with a first fluorescent dye to distribute to the vascular and interstitial spaces and a large dextran (molecule size of about 70 kDa to about 500 kDa, preferably about 70 kDa to about 250 kDa), labeled with a second fluorescent dye for distribution only to the vascular space of the animal. The molecules can be simultaneously detected in vivo using a dual channel fluorescence detection device and a proprietary fiber optic catheter. The fluorescence device and the fiber optic catheter are disclosed in pending U.S. patent application Ser. No. 12/425,827, the disclosure of which is hereby incorporated by reference as if fully set forth herein and, more specifically, for this specific subject matter disclosed at Paragraphs [0077] to [0093] and FIGS. 1 and 91-14 for the detector and Paragraphs [0108] to [0112] and FIGS. 1, 16, and 17 for the fiber optic catheter.

The method comprises: (1) inserting the proprietary fiber optic catheter into a peripheral vein in the patient's upper extremity; (2) connecting the fiber optic catheter to the fluorescence device; (3) attaching a syringe containing 5 to 10 ml of an injectate containing the small and large fluorescent dextrans to the catheter; (4) injecting 1 ml of the injectate into the calibration chamber of the catheter, and backfilling with patient's blood; (5) calibrating the fluorescence detection device; (6) advancing the fiber optic line through the syringe and into the catheter; (7) allowing enough time (approximately 10 to 15 minutes) for the molecules to equilibrate in the patient; (8) detecting the fluorescence intensities of the small and large dextrans with the fluorescence device; (9) calculating the fluid volumes using a pre-programmed algorithm; and (10) displaying the values of the fluid volumes on a screen.

Some of the key advantages of this method are that it is fast (only takes about 15 minutes), accurate and inexpensive. More importantly, the fluid volumes can be determined using data from a single time point.

While the specific embodiments have been illustrated and described, numerous modifications come to mind without significantly departing from the spirit of the invention, and the scope of protection is only limited by the scope of the accompanying claims.

What is claimed is:

1. A method for measuring total vascular plasma volume (TVPV) of an animal comprising:
(a) administering a sufficient amount (A) of a molecule to the vascular system of the animal, wherein the molecule is non-metabolized and impermeable to the vessel walls of the vascular system;
(b) allowing the molecule to reach an equilibrium steady state concentration in the plasma within the vascular system of the animal;
(c) measuring the equilibrium steady state concentration (C) of the molecule; and
(d) calculating the TVPV using the equation: TVPV=A/C;
wherein the molecule has a molecular weight of from about 70 kDa to about 250 kDa and comprises the conjugation product of: (i) a dextran, and (ii) a fluorescent rhodamine dye containing a single functional group, and
wherein the conjugation product further comprises a single isomer having an excitation wavelength and an emission wavelength, said isomer being capable of detection and measurement by its emission fluorescent intensity.

2. The method of claim 1 wherein the molecule has a molecular weight of about 150 kDa.

3. The method of claim 1 wherein the administration of the molecule is by intravenous injection of an injectate containing the molecule.

4. The method of claim 1 wherein the injection is a bolus injection or an infusion.

5. The method of claim 1 wherein the administration is by inhalation.

6. The method of claim 1 wherein the step (c) of measuring C further includes:
(a) withdrawing a sample of blood from the vascular system of the animal;
(b) obtaining a plasma supernatant from the blood sample; and
(c) measuring C in the supernatant of the sample.
